(19) United States
(12) Patent Application Publication
(10) Pub. No.: US 2025/0334597 A1
(43) Pub. Date: Oct. 30, 2025

(54) Sequential sample adding device and automatic sequential sample adding system

(71) Applicant: Zhejiang Attainose Medical Technology Co.,Ltd, Hangzhou (CN)

(72) Inventor: Shiping SONG, Hangzhou (CN)

(73) Assignee: Zhejiang Attainose Medical Technology Co.,Ltd, Hangzhou (CN)

(21) Appl. No.: 18/857,191

(22) PCT Filed: Mar. 31, 2023
(86) PCT No.: PCT/CN2023/085398

§ 371 Date: Jul. 18, 2025

(30) Foreign Application Priority Data
Apr. 19, 2022 (CN) 202210407816.0

(51) Int. Cl. G01N 35/00; G01N 27/327; G01N 30/64; G01N 33/543; G01N 33/569

(52) U.S. Cl. CPC G01N 35/00029; G01N 27/3276; G01N 30/64; G01N 33/5438; G01N 33/56988; G01N 2035/00039; G01N 2469/10

(57) ABSTRACT

Provided is a sequential sample adding device and an automatic sample adding system. The sequential sample adding device includes a sample adding tube (1), N storage spaces are arranged in the sample adding tube (1) from one end to the other end. Each storage space comprises a solution section (4) and a piston (5), and the piston (5) can be in contact with a piston rod (6) to be pushed. A tube wall of the sample adding tube (1) is provided with a first liquid outlet (2) and N-1 second liquid outlets (3), which can be in communication with solutions inside the sample adding tube (1), and N-1 second liquid outlets (3) are respectively disposed at the piston section of the first storage space to N-1ₜₕ storage space.

### FIELD OF TECHNOLOGY

The present disclosure relates to the technical field of solution sample adding devices, especially to a sequential sample adding device and automatic sample adding system.

### BACKGROUND

In various chemical and biological experiments, human operational errors may lead to substantial discrepancies in the results. Therefore, minimizing such errors during experimental procedures stands as one of the impetuses for the advancement of related technologies. The workflows of chemical and biological experiments involving solutions typically encompass multiple sequential steps of liquid addition. The accurate control of both time and the volume of liquid added not only places greater demands on the operators but also heightens the susceptibility to human errors during the operational process.

The development of fully automatic experimental technologies and apparatuses is one of the approaches to solving human errors. For example, fully automatic biochemical analyzers and fully automatic immunity analyzers have emerged in the field of laboratory medicine. These have expedited medical testing procedures and enhanced the accuracy of results. However, fully automatic experimental equipment is not suitable for some experimental scenarios (e.g., on-site examinations) due to the large volume and weight caused by containing several mechanical modules necessary for automatic design. Also, the batch experiment capacity thereof is not suitable for single experimental operations, otherwise it will cause great waste. In addition, the fully automatic equipment typically requires a relatively large amount of solution, which is not suitable for micro-experimental operation.

Chip technology founded on microfluidic structures is suitable for micro-operations. However, it encounters numerous challenges in the aspect of automation, especially in the case of sequential sample adding. Microfluidic chip, also known as “lab-on-a-chip”, is a novel technology for integrating a plurality of experimental steps in a small-volume device. Although the microfluidic chip utilized for experimental executions is of a diminutive size per se, it needs external solution input and auxiliary operation equipment, such as equipment for facilitating the solution flow (e.g., syringe pumps, peristaltic pumps, or centrifuges, etc.), solution switching devices (micro-valves such as bubbles, and corresponding pipelines) and equipment for controlling the flow rate, etc. If a microfluidic chip is required to achieve sequential sample adding, one method is to manually substitute different solutions at the solution inlet end, and subsequently pump them in via a syringe pump or a peristaltic pump, or draw them in by negative pressure generated by the centrifuge at the terminus. Given that the solution replacement demands manual manipulation, bubbles will inevitably be introduced during the replacement process. An alternative method is to set an automatic switching device at the solution inlet end, e.g., using an automatically controlled fluid multi-way valve to switch one liquid to another automatically. Nevertheless, it is also difficult to completely avoid the introduction of bubbles with this method. Moreover, the internal volume of the microfluidic chip structure is extremely small, which is mainly suitable for liquid experiments at the microliter scale, rather than those at the milliliter scale or above.

Because the operation processes require multiple sequential sample adding of liquids, there exists a demand for automatic and accurate sequential sample adding.

CN205163822U discloses a pre-filled segmented syringe, which can be pre-filled with solutions in different segments to realize sequential sample adding. The syringe includes a syringe needle head part and a syringe needle tube part. The syringe needle tube part includes a plurality of first-stage needle tubes, second-stage needle tubes, and multi-stage needle tubes which are sleeved with each other from top to bottom. The bottoms of both the second-stage needle tubes and the multi-stage needle tubes are provided with pistons that can perform an up-and-down piston motion in the next-stage needle tubes, together with the corresponding stage needle tubes. The pistons at the bottoms of both the second-stage needle tubes and the multi-stage needle tubes are provided with sealing devices. The inner walls of the second-stage needle tubes and the multi-stage needle tubes are provided with locking mechanisms matched with the outer walls of the previous-stage needle tubes. The syringe needle head part includes injection needles arranged by penetrating the bottom of the first-stage needle tubes. The sealing device is arranged vertically above the injection needle. After the injection of the medicine in the bottommost needle tube is completed, the injection needle arranged by penetrating the bottommost syringe will pierce the sealing device at the bottom of the previous syringe so that the medicine in the syringe can be injected. Subsequently, repeat the above operations to achieve the technical effect of segmented propulsion. However, because there is only one outlet in this scheme, when an automatic sequential sample addition at different sites is needed, it cannot be accomplished only via the single outlet, and an additional automatically controlled fluid multi-channel valve and pipeline are still needed subsequently for cooperation. At the same time, when different solutions are sequentially released based on the segmented propulsion to achieve a sequential sample addition, the outlets share one channel, which will cause the problem of solution contamination.

Therefore, how to encapsulate two or more liquid solutions in multiple sections of a device and release the solutions respectively according to the requirements of sequential sample adding, and how to release the liquids in different sections at set times by controlling the movement of the propulsion members in the sequential sample adding device is still a problem in practice.

### SUMMARY

The present disclosure provides a sequential sample adding device and automatic sample adding system.

The sequential sample adding device includes a sample adding tube having a tube wall. The N storage spaces are provided in the sample adding tube from a first end to a second end, each of the N storage spaces includes a solution section and a piston section, wherein the solution section (4) is configured to accommodate a solution and the piston section is configured to accommodate a piston. The piston is located at the second end relative to the solution section, and N is greater than 1. The tube wall is provided with a first liquid outlet and N-1second liquid outlets, the first liquid outlet is in communication with a solution section of a first storage space. The first liquid outlet is pre-sealed, and N-1second liquid outlets are respectively disposed at the piston section of the first storage space to a N-1ₜₕ storage space. The piston is configured to separate solutions added in different storage spaces, push the solution section, and let the solutions added in different storage spaces flow out of liquid outlets or not. An end of the piston in a piston section of a storage space that is exposed to the outside is configured to contact a power device. When a sequential sample adding is performed, the first liquid outlet that is pre-sealed is opened, and the end of the piston in the piston section of the storage space is pushed by the power device so that a solution added in the solution section of the first storage space is released. A piston in a previous storage space moves towards the first end and makes solution sections of subsequent storage spaces be in communication with corresponding second liquid outlets, thus solutions added in the solution sections of the subsequent storage spaces are able to be sequentially released via the corresponding second liquid outlets when the end of the piston in the piston section of the storage space is then pushed. The solution cannot flow out without pushing due to the external air pressure. The first liquid outlet is pre-sealed so that the solution section of the first storage space cannot be in communication with the outside via the first liquid outlet.

The method for pre-loading the liquid solution into the sample tube may be to form an opening outside the tube wall, inject the solution via the opening, and then seal the opening.

In some embodiments, the power device can be driven by human hands, or it can be driven by air pressure, hydraulic pressure, or a motor, as long as it can push the piston to move. By the movement of the piston, the liquid solutions can be respectively released via the sample adding ports according to the requirements of sequential sample adding. If the device is driven by air pressure, hydraulic pressure, or a motor, the time intervals, propulsion rate, etc. of the driving can be controlled by a controller, so that an automatic release of the solutions to be added for the reaction can be realized according to the set sequence and time interval. In some embodiments, the power device is a motor drive device including a piston rod and a driver, an end of the piston rod is connected to the driver. The driver can move linearly and push the piston rod to drive the piston to move in a direction toward the solution section correspondingly.

In some embodiments, two or more liquid solutions are pre-loaded in the sample adding tube, and only one manual sample adding (manually adding the experimental sample to be tested) is needed at most when detecting an experimental sample. The pre-loaded liquid solutions can be automatically pushed by the power device, which greatly improves the convenience of the operation process. The sequential sample adding device of the present disclosure is suitable for making portable detection equipment, and more suitable for on-site detection. If the pre-loaded liquid solutions are different, the design of the present disclosure allows two or more different solutions to be added completely independently with no pipeline contamination.

In certain embodiments, the N storage spaces are sequentially arranged from the first end to the second end in one whole sample adding tube, the first liquid outlet and the N-1 second liquid outlets are arranged at intervals. The N-1second liquid outlets are respectively located at the piston sections of the first storage space to N-1ₜₕ storage space, and the piston in the piston section of Nth storage space contacts the power device.

In some embodiments, the sample adding tube is separated by piston(s) into two or more storage spaces which are connected in series. In the initial state, the first liquid outlet is in a pre-sealed state. The first liquid outlet can be pre-sealed with external air pressure, and can also be sealed by valves, blocks, plugs, and other sealing covers, or the first liquid outlet can be covered in other removable seals (such as removable pistons). N-1 second liquid outlets are respectively located within an area sealed by the piston in the previous storage space, and are not in communication with the subsequent storage space in the initial state. When the sequential sample adding begins, the sealing of the first liquid outlet is first removed to let the solution section of the first storage space be in communication with the outside. The piston is pushed by the power device so that the solution in the solution section of the first storage space is released via the first liquid outlet. When the piston is pushed to a specific position, the second liquid outlet covered by the piston is exposed to the solution in the corresponding subsequent storage space, so that the solution in the subsequent storage space can be released via the second liquid outlet. Various liquid outlets are sequentially exposed so that the two or more solutions required for the reaction can be automatically added to set areas according to a set order and time interval, which can greatly improve the automation degree of the reaction.

In certain embodiments, the sample adding tube comprises N independent chambers arranged side by side, and the N storage spaces are respectively located inside the N independent chambers. Each independent chamber comprises a tube wall, a solution section, and a piston section. A tube wall of a first independent chamber is provided with a first liquid outlet that is in communication with the solution section of the first independent chamber, and a second independent chamber to Nth independent chamber are respectively provided with a second communication port on their tube wall, wherein the second communication port is in communication with one corresponding solution section of the second independent chamber to Nth independent chamber, and the first liquid outlet is pre-sealed. Each independent chamber further comprises a first communication port and a second liquid outlet adjacently arranged. The first communication port and the second liquid outlet are located at the piston section. The second liquid outlet is not in communication with the first communication port when a piston is provided in the piston section, and the second communication ports of the second independent chamber to the Nth independent chamber are correspondingly in communication with the first communication ports of the first independent chamber to N-1ₜₕ independent chamber via communication tubes. A groove is axially provided on an outer surface of the piston arranged in each independent chamber at an end away from the solution section. When the piston is pushed, the groove moves accordingly, and when the groove arrives at a position where both the first communication port and the second liquid outlet are covered, the first communication port is in communication with the second liquid outlet via the groove, and the second liquid outlet is in communication with the solution section of a subsequent independent chamber.

In some embodiments, an end of each piston is configured to contact a power device, and each power device respectively pushes the corresponding piston. Such independent chambers arranged side by side have more reasonable overall lengths. If the front end of the powder device is a piston rod, it can avoid the technical problem that the length of the piston rod extending out of the sample adding tube is too long in serial mode. The drivers of various pistons can be driven independently, or can be linked by programming, to achieve an automatic sequential sample adding with high throughout.

In addition, since each independent chamber is a relatively independent reaction unit, the communication tube between two independent chambers can be cut off as needed, and the second communication port of the subsequent independent chamber can become a new first liquid outlet. It allows the independent chambers arranged side by side to be split into multiple sequential sample adding devices, and it is helpful for the operator to handle flexibly.

In any embodiment of the present disclosure, the first liquid outlet and the N-1 second liquid outlets can be respectively connected to liquid outlet tubes. Such independent setting of the liquid outlet tubes ensures no mutual contamination between different solutions, and further helps to direct the liquid outlet tubes to the sample adding inlets at different positions, thereby meeting the demands of adding different solutions at different sample adding positions. For example, it can add different solutions at different positions in some chips with a microfluidic structure or a chromatographic structure.

In some embodiments, the liquid outlet tube is provided with a retention chamber for accommodating the solution. The outlet of the liquid outlet tube is connected correspondingly to the inlet of the solution-receiving element. The retention chamber can avoid the occurrence of capillary effect at the solution- receiving element and the formation of siphon phenomenon.

The present disclosure further discloses an automatic sequential sample adding system, including the aforesaid sequential sample adding device, a piston rod, and a driver. In some embodiments, the driver is driven by a stepping motor, and the outlet of the stepping motor is connected to a ball screw. The ball screw is provided with a lead screw nut, and the end surface of the lead screw nut is in contact with the piston rod. The stepping motor is connected to a controller which intermittently pushes the piston rod to move by a delay control of the lead screw nut. The movement of the stepper motor is controlled by the controller, including controlling the time interval for initiating the propulsion and the propulsion rate. The solutions required for the reaction are automatically added to the set areas according to a set order and time interval, which greatly improves the automation degree of the reaction. By releasing the solutions at different positions at the set times via the piston, the manual operation errors are eliminated, to greatly improve the accuracy of the reaction results. The controller may be a programmable controller PLC, for example.

The sequential sample adding device of the present disclosure can be applied to various scenarios that require sequential sample adding, which can be pushed manually or automatically. For example, it can be used together with a detection chip with a chromatographic structure or a microfluidic structure that requires sequential sample adding. Therefore, the present disclosure further provides an automatic detection system for sequential sample adding, including the sequential sample adding device, a power device, and a detection device. The first liquid outlet and the N-1 second liquid outlets of the sequential sample adding device are respectively connected to different sample adding inlets of the detection device, and the power device automatically drives the pistons of the sequential sample adding device so that solutions in the solution section are sequentially released. In some embodiments, the detection device is a detection chip with a chromatographic structure or a microfluidic structure.

Some detection chips with a chromatographic structure, e.g., chromatographic electrochemical biochips for pesticide detection or immunoassays, need multiple times of sequential sample adding when using. Taking the chromatographic electrochemical biochip for the immunoassay of AIDS antibody Ab (HIVAb) as an example, an HIV antigen (S-HIVAg) is immobilized on its working electrode as detection area, and an HIV antigen-horseradish peroxidase (HIVAg-HRP) is sprayed and adsorbed onto the front end of the chromatographic test strip, the HIVAg-HRP spraying area receives the sample adding of HIV antibody (HIVAb) to be tested and the sample adding of washing solution. The detection area where the working electrode is located receives the substrate TMB solution. Among them, the HIV antibody sample needs to be manually added, and subsequently, the washing solution and the substrate TMB solution need to be added in sequence, with an interval of about ten minutes. The washing solution can fully elute other substances unbinding to the HIV antigen (S-HIVAg) in the detection area by chromatographic effect, allowing those substances to move out of the detection area and leave “Ag-Ab-Ag (S-HIVAg-HIVAb-HIVAg-HRP)” type sandwich conjugate. Then, the substrate TMB is added to undergo an enzymatically catalyzed reaction with the HRP to output a current signal.

Some detection chips with microfluidic structures, e.g., chromatographic electrochemical biochips for pesticide detection or immunoassays, need multiple times sequential sample adding when using. Taking the microfluidic electrochemical biochip for the immunoassay of AIDS antibody (HIVAb) as an example, an HIV antigen (S-HIVAg) is immobilized on its working electrode as a detection area. The HIV antibody sample needs to be manually added, and subsequently, the primary washing solution, the HIV antigen-enzyme complex (HIVAg-HRP), the secondary washing solution, and the TMB substrate solution need to be added in sequence, with an interval of about ten minutes. The primary washing solution can fully wash other substances unbinding to the HIV antigen (S-HIVAg) out in the detection area by dynamic microflow, allowing those substances to move out of the detection area. Then, the HIV antigen-enzyme complex (HIVAg-HRP) moves into the detection area by dynamic microflow, and binds to the captured HIV antibody in the detection area, forming an “Ag-Ab-Ag (S-HIVAg-HIVAb-HIVAg-HRP)” type sandwich complex. The secondary washing solution fully elutes the uncaptured HIV antigen-enzyme complex by dynamic microflow. Then the substrate TMB is added to undergo an enzymatically catalyzed reaction with the HRP to output a current signal.

### BRIEF DESCRIPTION OF THE DRAWINGS

FIG. 1 is a schematic diagram of an internal installation structure of a sequential sample adding device in Example 1 of the present disclosure.

FIG. 2 is an enlarged view of A in FIG. 1.

FIG. 3 is a diagram of the change in the usage state of Example 1 of the present disclosure.

FIG. 4 is a diagram of the change in the usage state of Example 2 of the present disclosure.

FIG. 5 is an enlarged view of B in FIG. 4.

FIG. 6 is an enlarged view of C in FIG. 4.

FIG. 7 is a schematic diagram of the structure of a liquid receiving tube in Example 4 of the present disclosure.

FIG. 8 is a structural diagram of a chromatographic electrochemical biochip cooperating with Example 1 of the present disclosure.

FIG. 9 is a diagram showing the test results of a chromatographic electrochemical biochip cooperating with Example 1 of the present disclosure.

### REFERENCE NUMERALS

1. Sample adding tube;
2. First liquid outlet;
3. Second liquid outlet;
4. Solution section;
5. Piston;
6. Piston rod;
7. First liquid outlet tube;
8. Communication tube;
9. Second liquid outlet tube;
10. Retention chamber;
51. Groove;
81. First communication port;
82. Second communication port;
A. Front end of the chip;
B. Rear end of the chip;
11. Working electrode;
21. Sample pad;
22. Chromatographic membrane;
23. Absorbent pad;
24. HIVAg-HRP sprayed area;
25. Detection area;
31. Bottom cover;
32. Top cover;
321. Sample adding hole for substrate release;
322. Sample adding hole for HIV antibody loading and washing solution release;
33. Front opening.

### DETAILED DESCRIPTION

Hereinafter the present disclosure is further described with reference to the accompanying drawings.

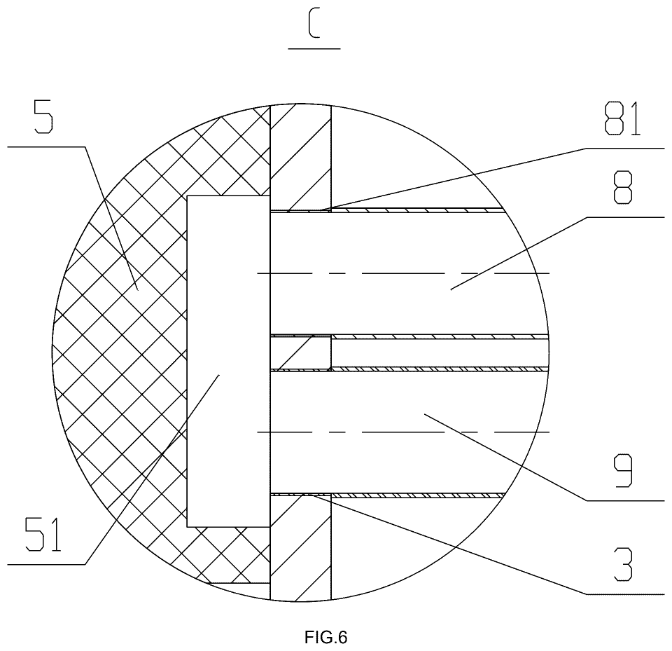

Referring to FIGS. 1-9, the structures, proportions, sizes, or the like as depicted in the accompanying drawings of the present disclosure are only used to cooperate with the disclosure of the description for the understanding and reading of persons skilled in the art, rather than to limit the conditions for practicing the present disclosure, and thus they have no technical substantive significance. Any modifications in structure, changes in proportions, or adjustments in size should still fall within the scope of the present disclosure in the case that the effect to be produced and the object to be achieved by the present disclosure are not affected. At the same time, terms such as “upper”, “lower”, “left”, “right”, “middle”, and “a/an” referenced in the present description are only used for the convenience, and are not used to limit the scope for implementation of the present disclosure. The changes or adjustments in their relative relationships shall also be regarded as the scope for implementation of the invention, as long as there is no substantial change in the technical content.

### Example 1

A sequential sample adding device is in serial mode. As shown in FIG. 1, the sample adding tube 1 is a whole tube having a tube wall. Four storage spaces are provided in the sample adding tube 1 from a first end to a second end, each storage space includes a solution section 4 and a piston section, the solution section 4 is configured to accommodate a solution, and the piston section is configured to accommodate a piston 5. The piston section is located at the second end relative to the solution section 4. The tube wall is provided with a first liquid outlet 2 and three second liquid outlets 3, the first liquid outlet 2 and three subsequent liquid outlets are in communication with the solutions in the corresponding solution section. The first liquid outlet 2 is pre-sealed by a plug (not shown). The three second liquid outlets are respectively disposed in the piston section of the first storage space to 3rd storage space. The pistons 5 is configured to separate solutions added in different storage spaces, push the solution section, and let the solutions added in different storage spaces flow out of liquid outlets or not. The piston in a piston section of the 4th storage space is configured to contact a power device.

The method for pre-loading the liquid solution into the sample tube is to form an opening on the upper part of the tube wall, inject the solution via the opening, and then seal the opening (not shown).

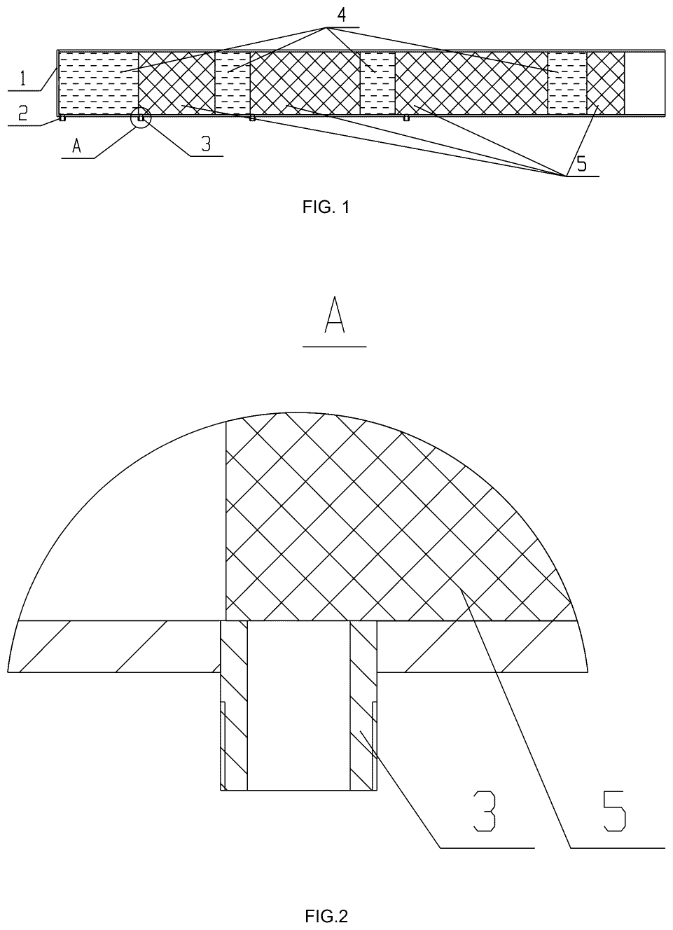

In addition to the pre-sealing of the first liquid outlet, as shown in FIG. 2, the lower end of each second liquid outlet on the tube wall is open to the outside. However, at the initial time, the upper end of each second liquid outlet is within the coverage of the piston. Thus, any second liquid outlet cannot be in communication with the solution section of the subsequent storage spaces.

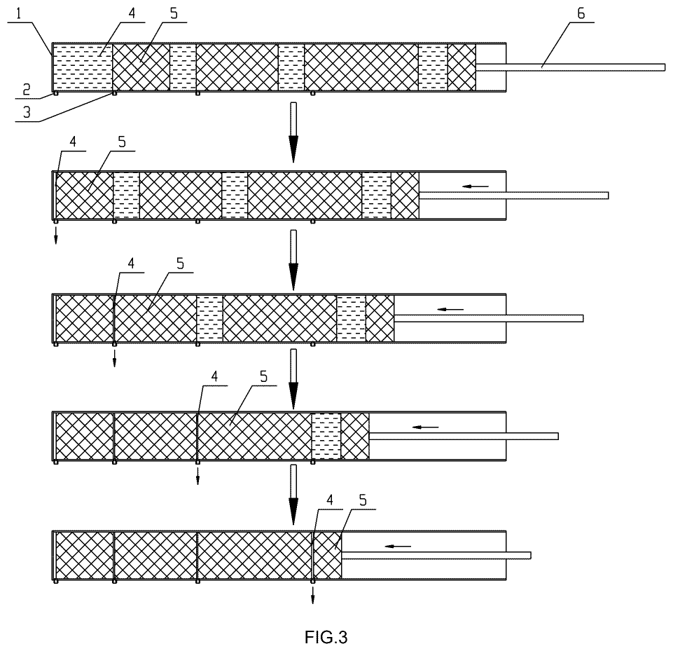

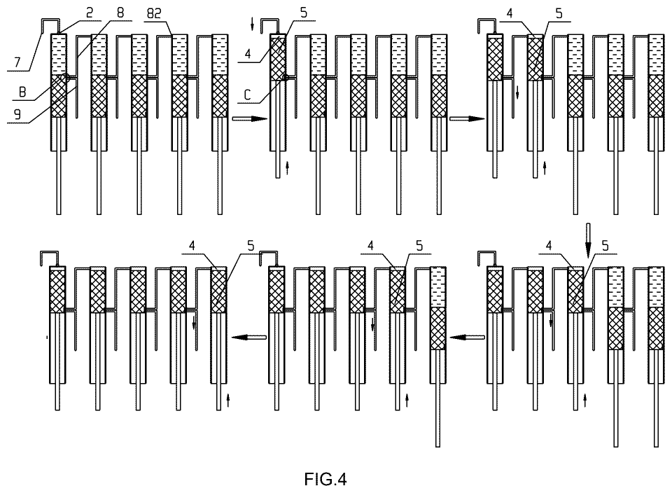

FIG. 3 shows the operation process of the sequential sample adding device in serial mode. At the beginning, the end of the piston 5 in the fourth storage space is in contact with the piston rod 6. The piston rod 6 can be pushed manually or driven by a stepper motor (not shown). When a sequential sample adding is performed, the pre-sealed first liquid outlet 2 is opened. The piston 5 is pushed by the piston rod 6, the solution and the piston in the tube as a whole move forward, the solution in the solution section 4 of the first storage space is released via the first liquid outlet 2, and the second liquid outlet 3 is exposed to the solution section 4 in the second storage space due to the forward movement of the piston 5 in the first storage space. At that time, the pushing stops, and the solution in the solution section of the second storage space will not be released due to the atmospheric pressure, although the second liquid outlet 3 has been in communication with the solution section 4 of the second storage space. Until the pushing re-begins, the solution in the solution section of the second storage space will be released via the second liquid outlet 3. In this way, the piston 5 of the previous storage space moves to a position where the subsequent solution section 4 is in communication with the corresponding subsequent liquid outlet, so that the subsequent solution section 4 can be released via the subsequent liquid outlet 3 when pushed. The solutions in various solution sections can be sequentially released, thereby the sequential sample adding can be achieved. If a stepping motor is employed for driving, the outlet of the stepping motor is connected to a ball screw provided with a lead screw nut, and the end surface of the lead screw nut is in contact with the piston rod. The stepping motor is connected to a controller which intermittently pushes the piston rod 6 to move by a delay control of the lead screw nut. By programming the controller, the pushing process can be accurately positioned and controlled, and the interval between sample adding of different solutions can also be accurately controlled.

As shown in FIGS. 1 to 3, a thread or buckle (not shown) can be arranged outside the liquid outlet, to facilitate a tight connection between the liquid outlet and the liquid outlet tube.

The present embodiment provides a sequential sample adding device including four storage spaces. According to the actual needs, the number of storage spaces can be two or more.

Two or more liquid solutions are pre-loaded in the sample adding tube, by controlling the pushing process and time interval of the piston rod, the piston rod is driven by a driver to move so that the liquid solutions can be released via the sample adding ports according to the requirements of the sequential sample, thereby achieving a precise sequential sample adding. It greatly improves the degree of convenience of the reaction procedure, and it is suitable for making a portable device, and is more suitable for on-site detection applications.

Two or more liquid solutions can be the same or different. When the solutions in the solution sections are different, the different liquid outlets can be connected respectively, which cannot only avoid contamination between solutions, but also direct different solutions to different positions. It avoids additional complex pipelines, and can achieve automatic sequential sample adding in different areas by piston pushing.

### Example 2

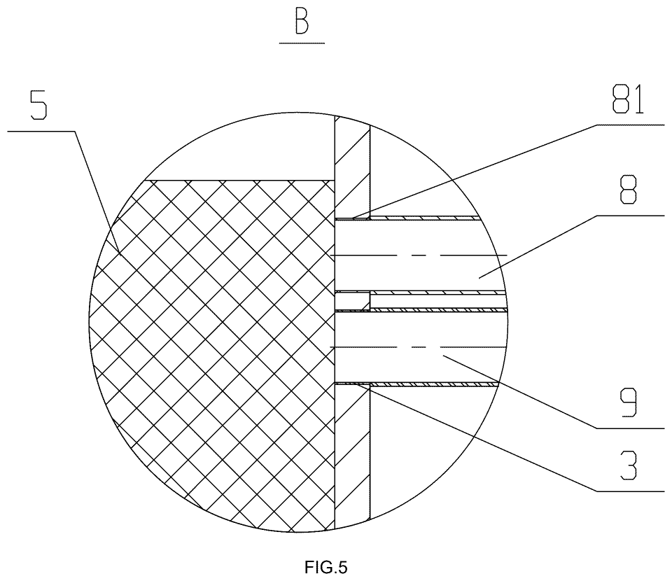

A sequential sample adding device is in parallel mode. As shown in FIGS. 4, 5, and 6, the sample adding tube 1 includes five independent chambers arranged side by side. Five storage spaces are respectively provided in the five independent chambers, each independent chamber includes a tube wall, and a solution section 4 and a piston section are provided in the chamber. This embodiment adopts independent chambers arranged side by side and has a more reasonable overall length. It avoids the technical problem that the length of the piston rod extending out of the sample adding tube is too long in the presence of many storage spaces in serial mode. As shown in FIG. 4, the tube wall of the first independent chamber (the far left) is provided with a first liquid outlet 2 in communication with the solution section 4 of the first independent chamber, and a second independent chamber to the 5th independent chamber are provided with second communication ports 82 on their tube wall, and the second communication ports 82 are in communication with the upper ends of the solution sections 4 of the second independent chamber to 5th independent chamber. The first to the fourth independent chambers further include first communication ports 81 and second liquid outlets 3 adjacently arranged. The first communication ports 81 and the second liquid outlets 3 are located at the piston section, and the second liquid outlets 3 are not in communication with the first communication ports 81 in the initial state. The second communication ports 82 of the second to the fifth independent chambers are in communication with the first communication ports 81 of the first independent chamber to the N-1ₜₕ independent chamber via communication tubes 8. A groove 51 is axially provided on an outer surface of the piston 5 arranged in each independent chamber at an end away from the solution section 4. The length of the grooves 51 can cover both the first communication ports 81 and the second liquid outlets 3. The first liquid outlet 2 is pre-sealed in its initial state, and it can be unsealed when using.

An end of the piston 5 in any independent chamber is configured to contact the piston rod 6 respectively provided, and each piston rod 6 can be connected to a corresponding driver.

In this embodiment, the first liquid outlet 2 is in communication with the first liquid outlet tube 7, to direct the solution to the area where a sample adding is required. A valve (not shown) can be used to connect the first liquid outlet tube 7 and to achieve pre-sealing.

FIG. 4 shows the operation process of the sequential sample adding device in parallel mode. When a sequential sample adding is carried out, the valve is rotated so that the first liquid outlet 2 is in communication with the first liquid outlet tube 7, and the piston rod 6 of the first independent chamber is driven to push the piston 5 so that the solution section 4 at its upper end is released outward via the first liquid outlet 2 and the first liquid outlet tube 7. At the end of the solution release, the groove 51 on the piston 5 moves to a position where the first communication port 81 and the second liquid outlet 3 are covered, so that the first communication port 81 is in communication with the second liquid outlet 3 via the groove 51, and thus in communication with the solution section 4 of the subsequent independent chamber via the communication tube 8. When the piston 5 in the subsequent independent chamber is pushed by its corresponding piston rod 6, the solutions in its corresponding solution section 4 are released via the second communication port 82 of the subsequent independent chamber, the communication tube 8, the first communication port 81 of the previous storage space, the groove 51, and the second liquid outlet 3 in sequence. The second liquid outlet 3 is connected to the corresponding second liquid outlet tube 9 to direct to an area where a sample adding is needed. By operating in sequence, the liquid in the five independent chambers can be released in sequence.

### Example 3

Based on Example 1 or Example 2, an automatic sequential sample adding system includes the sequential sample adding device, a piston rod 6, and a driver. The driver includes a stepping motor, the outlet of the stepping motor is connected to a ball screw provided with a lead screw nut, and the end surface of the lead screw nut is in contact with the piston rod 6. The stepping motor is connected to a controller which intermittently pushes the piston rod 6 to move by a delay control of the lead screw nut. In the present embodiment, the movement of the stepper motor is controlled by the controller and includes the time interval for initiating the propulsion and the propulsion rate, so that the solutions required for the reaction are automatically added to the set areas according to the set order and time interval. It greatly improves the degree of automation of the reaction. By releasing the liquids at different positions at the set times using the pistons, the manual operation errors are eliminated, to greatly improve the accuracy of the reaction results. The screw nut can be fixedly connected to the piston rod 6 to prevent the piston rod from driving the piston due to accidental movement.

### Example 4

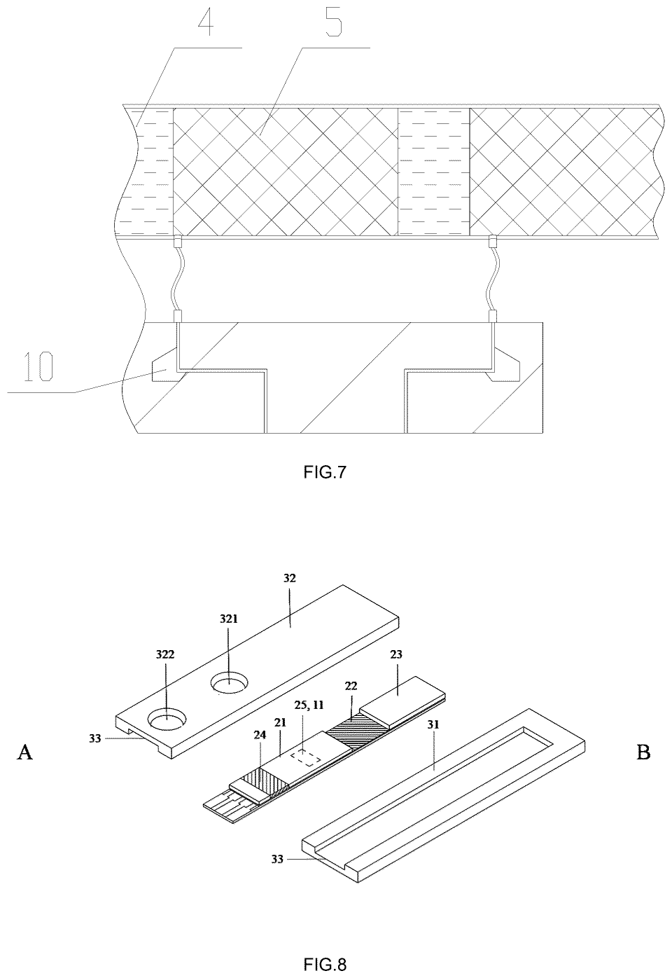

As shown in FIG. 7, the first liquid outlet 2 and the second liquid outlet 3 can be respectively connected to a liquid receiving tube. The middle of the liquid receiving tube is provided with a retention chamber 10 for accommodating the solution, and an outlet of the liquid receiving tube is connected correspondingly to an inlet of the solution-receiving element.

### Example 5

An experiment on the detection of AIDS virus (HIV) antibodies by chromatographic electrochemical double-antigen sandwich method is realized by the sequential sample adding device.

Experimental reagents include: HIV antigen (HIVAg) (Guangdong Fapon Biotech Co., Ltd.); horseradish peroxidase (HRP)-labeled HIV antigen (HIVAg-HRP) (Guangdong Fapon Biotech Co., Ltd.); HIV-I antibody (HIV-I Ab) (5 k) (Guangdong Fapon Biotech Co., Ltd.); Bovine serum albumin (BSA) (Sangon Biotech Shanghai Co., Ltd.); Casein (Sigma); 3,3′,5,5′-Tetramethylbenzidine (TMB) substrate solution (0.4 g/L) (Thermo company); 1*PBS buffer solution; 1*PBST washing solution (500 μL of Tween-20 is added into 1 L of 1*PBS buffer); and sodium carbonate buffer solution (coating solution) with a pH of 9.6.

Experimental raw materials include: screen-printed electrode chip (Zhejiang Nazhihui Biotech Co., Ltd.), glass cellulose membrane (Shanghai Jieyi Biotechnology Co., Ltd., product model: GL0145), absorbent pad (Shanghai Jieyi Biotechnology Co., Ltd., product model: H5015), nitrocellulose membrane (Whatman, Whatman AE99).

As shown in FIG. 8, the experimental detection structure includes a chromatographic electrochemical detection chip, where A is the front end of the chip, and B is the back end of the chip. A top cover 32 is disposed above the chromatographic electrochemical detection chip, and a bottom cover 31 is disposed below the chromatographic electrochemical detection chip. The mating surfaces of both the top cover 32 and the bottom cover 31 are provided with rectangular open grooves, and the electrochemical detection chip is installed in the open grooves. The openings of the open grooves are both front openings 33. The top surface of the top cover 32 is provided with a sample adding hole for HIV I antibody sample adding and washing solution release 322 and a sample adding hole for substrate release 321 in sequence from front to back. The chromatographic electrochemical detection chip includes an electrode chip and a chromatographic test strip, and the chromatographic test strip is tightly adhered the electrode chip. The electrode chip includes a working electrode 11, a counter electrode and a reference electrode. The counter electrode and the reference electrode are respectively located on two sides of the working electrode 11, and the above three electrodes are produced on the substrate by the screen printing process to form the electrode chip. The leads of the three electrodes are gathered at the front end of the chip, and extend from the front end of the test strip. The HV antigen is fixed on the working electrode 11. The chromatographic test strip includes a sample pad 21, a chromatographic membrane 22, and an absorbent pad 23 sequentially stacked. An HIVAg-HRP sprayed area 24 is provided on a position close to the A end of the sample pad 21, a detection area 25 is located in the middle of the sample pad 21, and the working electrode 11 is located within the range of the detection area 25.

The experimental process includes the following steps.

1) HIV antigen (HIVAg) was diluted to 100 μg·mL⁻¹ with a coating solution (pH 9.6) to obtain a diluent. 6 μL diluent is added dropwise to the working electrode 11 of the electrode chip, and incubated overnight at 4° C.

2) The surface of the working electrode 11 is rinsed with 1*PBS to remove the coating solution, and dried over N₂. Then 50 μL of 1% BSA+1% Casein mixed blocking solution (formulated with 1*PBS) is added dropwise to the three-electrode area of the electrode chip, and the blocking condition is at 37° C. for 2 h. The electrode chip is rinsed with 1*PBS to remove the blocking solution, and blown to dry. At that time, the HIV antigen on the working electrode is a coated antigen, referred to as S-HIVAg.

3) The HIVAg-HRP is diluted with the blocking solution described in step 2) at a ratio of 1:200, sprayed at an end of the glass cellulose membrane sample pad 21 by a commercial film sprayer to form a sprayed area, and dried in a constant temperature and humidity incubator at 25° C. for 2 h.

Three materials including the glass cellulose membrane sample pad 21, the chromatographic membrane 22, and the absorbent pad 23 are stuck and fixed to the electrode chip of step 2), and overlapped at the junctions thereof to finally form the chromatographic electrochemical detection chip. Regarding the position relationship, the HIVAg-HRP sprayed area 24 is close to the A end, and the sample adding hole for HIV-I antibody sample adding and washing solution release 33 is directly above the HIVAg-HRP sprayed area 24. The sample pad 21 covers the working electrode 11, the corresponding covered area is the detection area 25, and the chromatographic membrane does not cover the electrode.

The 1*PBST washing solution and the HRP enzyme catalytic substrate TMB solution are pre-packaged in the sample adding tube 1 similar to the serial sequential sample adding device in Example 1. As shown in FIG. 1, the solution section 4 contained in the first storage space is 150 μL of the 1*PBST washing solution, followed by the first section piston 5. The solution section 4 contained in the second storage space is 100 μL of the substrate TMB solution, followed by the second section piston 5. The end of the second section piston 5 is in contact with the piston rod 6 (not depicted), and the screw for pushing the piston rod is driven by a programmable stepper motor. The first liquid outlet of the sample adding tube 1 corresponds to the sample adding hole 322 of the chip, and the second liquid outlet corresponds to the sample adding hole 321 of the chip.

The HIV-I antibody with a titer of 5 k is diluted with the above blocking solution at different multiples (1:1000, 1:500, 1:100, 1:20). 150 μL diluent is added dropwise to the sample adding hole 322 to form a conjugate of HIV-I antibody and HIVAg-HRP immunoreaction, which is transmitted to the detection area by chromatography, and conjugated with the HIV antigen (S-HIVAg) in the detection area, to form an “Ag-Ab-Ag (S-HIVAg-HIV-I Ab-HIVAg-HRP)” type sandwich conjugate.

After 15 minutes of the sample adding, the pre-sealing of the sample tube is released, and the stepper motor is controlled by a program to drive the piston 5 by pushing the piston rod, so that the pre-packaged 1*PBST washing solution is released via the first liquid outlet 2, and released to the test strip of the chromatographic electrochemical detection chip via the sample adding hole 322. At the end of the washing solution release, the second liquid outlet hole is exposed, and the push stopped. After 10 minutes, the piston rod is pushed again by program control to drive the piston so that the pre-packaged substrate TMB solution is released via the second liquid outlet, and released to the test strip detection area 25 corresponding to the working electrode 11 of the chromatographic electrochemical detection chip via the sample adding hole 321.

The above 10-min interval was adopted so that the washing solution can fully elute other substances unbinding to the HIV antigen (S-HIVAg) in the detection area by chromatography and allow them to move out of the detection area. The subsequently added substrate TMB undergo an enzymatically catalyzed reaction with the HRP, and the current data was collected by the time current curve (i-t). The test potential is −0.1V, and the current signal value collected at 50 s after the test time is the output detection data.

The test data of the HIV-free blocking solution is the blank background data.

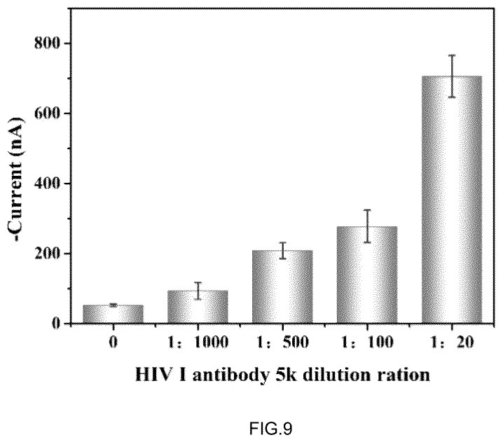

Result Analysis: HIV-I antibody is diluted at a ratio of 1:1000, 1:500, 1:100,and 1:20. The detection results are shown in FIG. 9. Even if the antibody is diluted at 1:1000, it can be clearly distinguished from the background signal.

In practice, the sequential liquid adding structure in serial mode and the sequential liquid adding structure in parallel mode of the present disclosure can be flexibly selected for sequential liquid adding, which can make the portable device adaptable to a variety of application scenarios.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims.

1. A sequential sample adding device, comprising a sample adding tube (1) having a tube wall,
wherein N storage spaces are provided in the sample adding tube (1) from a first end to a second end of the sample adding tube (1), each of the N storage spaces comprises a solution section (4) and a piston section, wherein the solution section (4) is configured to accommodate a solution and the piston section is configured to accommodate a piston (5), wherein the piston section is located at the second end relative to the solution section (4), and N is greater than 1;
wherein the tube wall is provided with a first liquid outlet (2) and N-1 second liquid outlets (3), the first liquid outlet (2) is in communication with a solution section (4) of a first storage space; the first liquid outlet (2) is pre-sealed so that the solution section (4) of the first storage space is not in communication with the outside via the first liquid outlet (2); N-1 second liquid outlets (3) are respectively disposed at the piston section of the first storage space to N-1ₜₕ storage space, wherein when the piston (5) is arranged in the piston section, the piston (5) is configured to separate solutions added in different storage spaces, push the solution section, and let the solutions added in different storage spaces flow out of liquid outlets or not;
wherein an end of the piston (5) in a piston section of a storage space that is exposed to the outside is configured to contact a power device; when a sequential sample adding is performed, the first liquid outlet (2) that is pre-sealed is opened, and the end of the piston (5) in the piston section of the storage space that is exposed to the outside is pushed by the power device, so that a solution added in the solution section (4) of the first storage space is released, and a piston (5) in a previous storage space moves towards the first end and makes solution sections (4) of subsequent storage spaces be in communication with corresponding second liquid outlets (3), thus solutions added in the solution sections (4) of the subsequent storage spaces are able to be sequentially released via the corresponding second liquid outlets (3) when the end of the piston (5) in the piston section of the storage space that is exposed to the outside is then pushed.

2. The device of claim 1, wherein the power device comprises a piston rod (6) connected to a driver that moves linearly, and the driver is configured to push the piston rod (6) to drive the end of the piston (5) in the piston section of the storage space that is exposed to the outside to move towards the first end.

3. The device of claim 1, wherein the N storage spaces are sequentially arranged from the first end to the second end in one whole sample adding tube (1), the first liquid outlet (2) and the N-1 second liquid outlets (3) are arranged at intervals; the N-1 second liquid outlets (3) are respectively located at the piston sections of the first storage space to N-1ₜₕ storage space; and the piston (5) in the piston section of Nth storage space contacts the power device.

4. The device of claim 1, wherein the sample adding tube (1) comprises N independent chambers arranged side by side, and the N storage spaces are respectively located inside the N independent chambers;
wherein each independent chamber comprises a tube wall, a solution section (4), and a piston section; a tube wall of a first independent chamber is provided with a first liquid outlet (2) that is in communication with the solution section (4) of the first independent chamber, and a second independent chamber to Nₜₕ independent chamber are respectively provided with a second communication port (82) on their tube wall, wherein the second communication port (82) is in communication with one corresponding solution section (4) of the second independent chamber to Nth independent chamber, and the first liquid outlet (2) is pre-sealed;
wherein each independent chamber further comprises a first communication port (81) and a second liquid outlet (3) adjacently arranged; the first communication port (81) and the second liquid outlet (3) are located at the piston section; the second liquid outlet (3) is not in communication with the first communication port (81) when a piston (5) is provided in the piston section; and the second communication ports (82) of the second independent chamber to Nth independent chamber are correspondingly in communication with the first communication ports (81) of the first independent chamber to N-1ₜₕ independent chamber via communication tubes (8);
wherein a groove (51) is axially provided on an outer surface of the piston (5) arranged in each independent chamber at an end away from the solution section (4); when the piston (5) is pushed, the groove (51) moves accordingly, and when the groove (51) arrives at a position where both the first communication port (81) and the second liquid outlet (3) are covered, the first communication port (81) is in communication with the second liquid outlet (3) via the groove (51), and the second liquid outlet (3) is in communication with the solution section (4) of a subsequent independent chamber.

5. The device of claim 4, wherein an end of each piston (5) is configured to contact a power device, and each power device is driven independently.

6. The device of claim 1, wherein the first liquid outlet (2) and the N-1 second liquid outlets (3) are respectively connected to a liquid outlet tube.

7. The device of claim 6, wherein the liquid outlet tube is provided with a retention chamber, the retention chamber is configured to accommodate a solution, and an outlet of the liquid outlet tube is connected correspondingly to an inlet of a solution-receiving element.

8. An automatic sequential sample adding system, comprising the device of claim 1, a piston rod (6), and a driver, wherein the driver comprises a stepping motor, an outlet of the stepping motor is connected to a ball screw, the ball screw is provided with a lead screw nut, and an end surface of the lead screw nut is in contact with the piston rod (6), the stepping motor is connected to a controller, and the controller intermittently pushes the piston rod (6) to move by a delay control of the lead screw nut.

9. An automatic detection system, comprising the device of claim 1, a power device, and a detection device, wherein the first liquid outlet (2) and the N-1 second liquid outlets (3) of the sequential sample adding device are respectively connected to different sample adding inlets of the detection device, and the power device automatically drives the pistons of the sequential sample adding device so that solutions in the solution sections are sequentially released.

10. The system of claim 9, wherein the detection device comprises a detection chip with a chromatographic structure or a microfluidic structure.
